(19) United States
(12) Patent Application Publication
(10) Pub. No.: US 2009/0112127 A1
(43) Pub. Date: Apr. 30, 2009

(54) NITINOL GUIDEWIRE

(75) Inventors: Karl KEATING, Galway (IE); Eamon Brady, Elphin (IE); David Vale, Galway City (IE); Marc Gianotti, Portola Valley, CA (US); John Neilan, Gort (IE)

Correspondence Address: Sughrue Mion-ABBOTT LABS, 2100 Pennsylvania Avenue, N.W., Washington, DC, 20037, US

(73) Assignee: SALVIAC LIMITED, Dublin 2 (IE)

(21) Appl. No.: 11/925,829

(22) Filed: Oct. 27, 2007

(51) Int. Cl. A61M 25/01

(52) U.S. Cl. 600/585

(57) ABSTRACT

A medical guidewire (1) suitable for advancement through a vasculature comprises a core element (2) extending along the length of the guidewire (1), a first coil (6), and a distal end cap (7). The core element (2) comprises a proximal portion (3), an intermediate portion (4), and a distal portion (5). The distal portion (5) is plastically deformable to facilitate steering of the guidewire (1). The first coil (6) is located radially outwardly of the core element (2). The first coil (6) extends along the distal portion (5) and part of the intermediate portion (4).

### INTRODUCTION

This invention relates to a medical guidewire. In one embodiment this invention relates to a medical guidewire suitable for advancement through a vasculature to facilitate exchange of one or more medical devices over the guidewire.

### STATEMENTS OF INVENTION

According to the invention there is provided a medical guidewire comprising:—

a core element extending along at least part of the length of the guidewire;
at least part of the core element being plastically deformable for steering of the guidewire.

Because the core element is plastically deformable, this enables a user to form the core element into a desired shape and/or configuration to assist steering of the guidewire during advancement through a vasculature. For example a portion of the core element may be formed into a curve.

In one embodiment of the invention the core element comprises a distal portion. The distal portion may be plastically deformable. The distal portion may be of stainless steel. The distal portion may taper proximally radially inwardly. The distal portion may taper distally radially inwardly.

In one case the core element comprises an intermediate portion extending proximally of the distal portion. The intermediate portion may be coupled to the distal portion. The intermediate portion may overlap the distal portion. The distal end of the intermediate portion may be located distally of the proximal end of the distal portion. The intermediate portion may taper distally radially inwardly. The intermediate portion may extend around at least part of the circumference of the distal portion. The intermediate portion may extend in a spiral. The distal portion may extend around at least part of the circumference of the intermediate portion. The distal portion may extend in a spiral.

In another embodiment the intermediate portion is formed integrally with the distal portion. The intermediate portion may be formed separately from the distal portion. The intermediate portion may be of the same material as the distal portion. The intermediate portion may be of a different material to the distal portion. The intermediate portion may be of a shape-memory material. The intermediate portion may be of Nitinol. The intermediate portion may be of stainless steel.

In another case the core element comprises a proximal portion extending proximally of the intermediate portion. The proximal portion may be coupled to the intermediate portion. The proximal portion may overlap the intermediate portion. The distal end of the proximal portion may be located distally of the proximal end of the intermediate portion. The intermediate portion may taper proximally radially inwardly. The proximal portion may be formed integrally with the intermediate portion. The proximal portion may be formed separately from the intermediate portion. The proximal portion may be of the same material as the intermediate portion. The proximal portion may be of a different material to the intermediate portion. The proximal portion may be of stainless steel.

### BRIEF DESCRIPTION OF THE DRAWINGS

The invention will be more clearly understood from the following description of some embodiments thereof, given by way of example only, with reference to the accompanying drawings, in which:—

FIG. 1 is a cross-sectional, side view of a medical guidewire according to the invention;

FIG. 2 is an enlarged cross-sectional, side view of part of the medical guidewire of FIG. 1;

FIGS. 3 to 6 are views similar to FIG. 2 of other medical guidewires according to the invention;

FIG. 7 is an enlarged cross-sectional, side view of another part of the medical guidewire of FIG. 1;

FIGS. 8 to 13 are views similar to FIG. 7 of other medical guidewires according to the invention;

FIG. 14 is an enlarged partially cross-sectional, side view of part of another medical guidewire according to the invention;

FIGS. 15 to 19 are views similar to FIG. 7 of other medical guidewires according to the invention;

FIG. 20 is a view similar to FIG. 14 of another medical guidewire according to the invention;

FIG. 21 is an enlarged cross-sectional, side view of part of another medical guidewire according to the invention;

FIG. 22 is an enlarged cross-sectional, side view of part of the medical guidewire of FIG. 19;

FIGS. 23 to 31 are views similar to FIG. 7 of other medical guidewires according to the invention;

FIG. 32 is an enlarged cross-sectional, side view illustrating assembly of part of a medical guidewire according to the invention; and

FIG. 33 is an enlarged cross-sectional, side view of the assembled part of the medical guidewire of FIG. 32.

### DETAILED DESCRIPTION

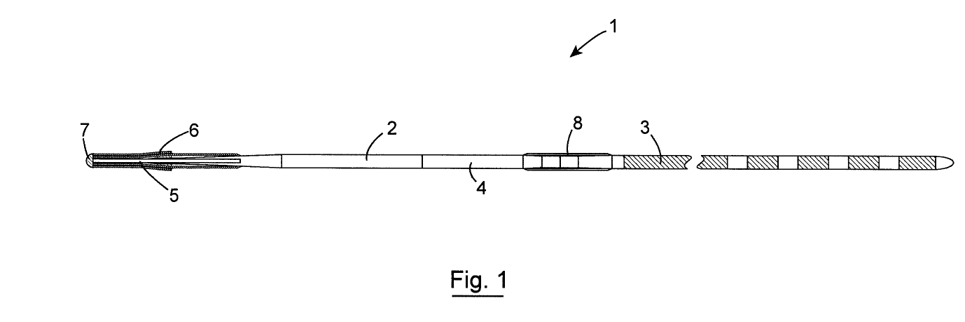

Referring to the drawings, and initially to FIGS. 1, 2 and 7 thereof, there is illustrated a medical guidewire 1 according to the invention. The guidewire 1 is suitable for advancement through a vasculature. One or more medical devices, for example a catheter or a stent or an embolic protection filter, may be exchanged over the guidewire 1.

The guidewire 1 comprises a core element 2 extending along the length of the guidewire 1, a first coil 6 and a distal end cap 7.

The core element 2 comprises a proximal portion 3, an intermediate portion 4 and a distal portion 5.

The distal portion 5 is plastically deformable to facilitate steering of the guidewire 1. In this case the distal portion 5 is of stainless steel.

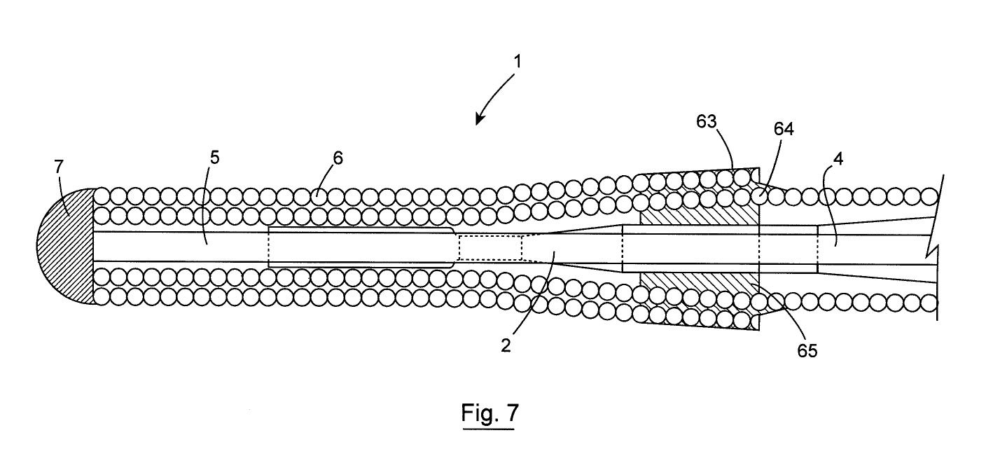

The distal portion 5 tapers proximally radially inwardly (FIG. 7).

The intermediate portion 4 is of a shape-memory material, for example Nitinol. The intermediate portion 4 is formed separately from the distal portion 5.

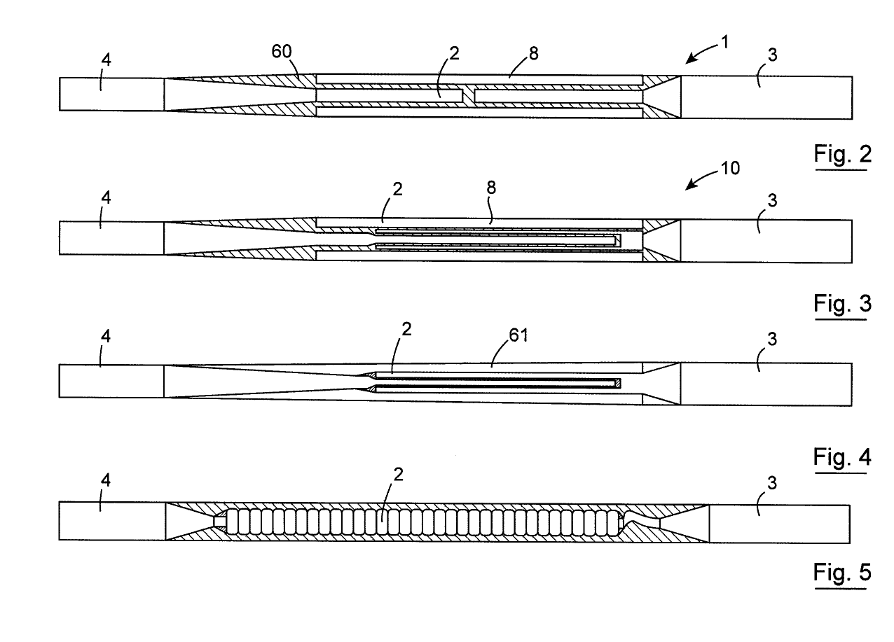

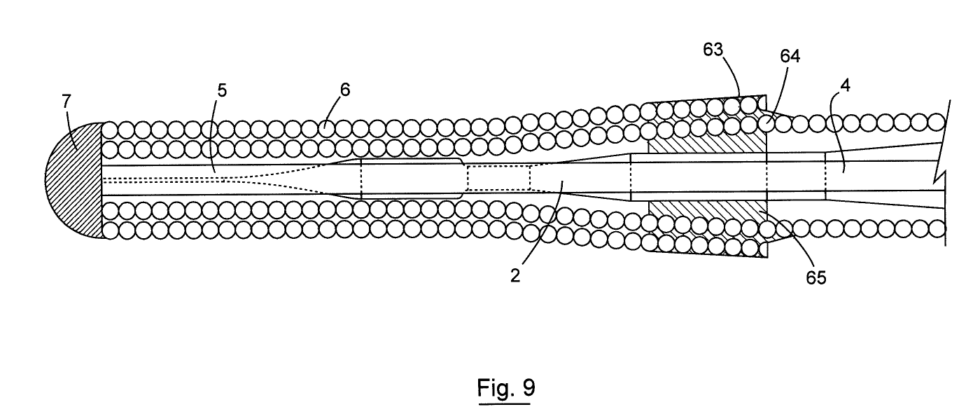

The intermediate portion 4 tapers distally radially inwardly (FIG. 9), and tapers proximally radially inwardly (FIG. 2).

The distal portion 5 is coupled to the intermediate portion 4 in an overlapping arrangement (FIG. 7). In particular the distal end of the intermediate portion 4 is located distally of the proximal end of the distal portion 5.

The intermediate portion 4 extends proximally of the distal portion 5.

The proximal portion 3 is of stainless steel, in this case. The proximal portion 3 is formed separately from the intermediate portion 4.

The proximal portion 3 tapers distally radially inwardly (FIG. 2).

The intermediate portion 4 is coupled to the proximal portion 3 by means of a hypotube assembly 8 (FIG. 2).

The proximal portion 3 extends proximally of the intermediate portion 4.

The first coil 6 is located radially outwardly of the core element 2 (FIG. 2). The first coil 6 extends along the distal portion 5 and part of the intermediate portion 4.

FIG. 1 illustrates the tip construction, and the SS proximal core 3 to NiTi distal core 4 coupling joint 8.

FIG. 2 illustrates the NiTi distal core 4, the adhesive bond 60, the NiTi hypotube 8, and the SS proximal core 3.

FIG. 7 illustrates the SS ribbon 5 which allows plastic deformation by the physician to provide tip steering characteristics, the NiTi core 4, solder 63 on the outer coils 6 which improves step integrity, the outer platinum coil 6 which provides a step, the inner SS coil 64 which helps build up the step, the adhesive bond 65. The distal end of the NITi core 4 is flattened to improve tip steerability.

In use, the distal portion 5 of the guidewire 1 is manipulated into a desired configuration, for example bent into a curve. The guidewire 1 is introduced into a vasculature and advanced through the vasculature until the guidewire 1 reaches a desired location in the vasculature. The curved distal portion 5 assists in steering of the guidewire 1. One or more medical devices may then be exchanged over the guidewire 1 to carry out a desired treatment procedure.

In FIG. 3 there is illustrated another medical guidewire 10 according to the invention, which is similar to the medical guidewire 1 of FIGS. 1, 2 and 7, and similar elements in FIG. 3 are assigned the same reference numerals.

In this case the intermediate portion 4 is coupled to the proximal portion 3 in an overlapping arrangement. In particular the distal end of the proximal portion 3 is located distally of the proximal end of the intermediate portion 4.

FIG. 3 illustrates the NiTi core 4 flattened and fit into a laser cut slot in the SS core 3, with the assembly bonded in place.

FIG. 4 illustrates a polymer surface 61.

FIG. 5 illustrates the SS core 3 coiled around the NiTi core 4 and bonded in place.

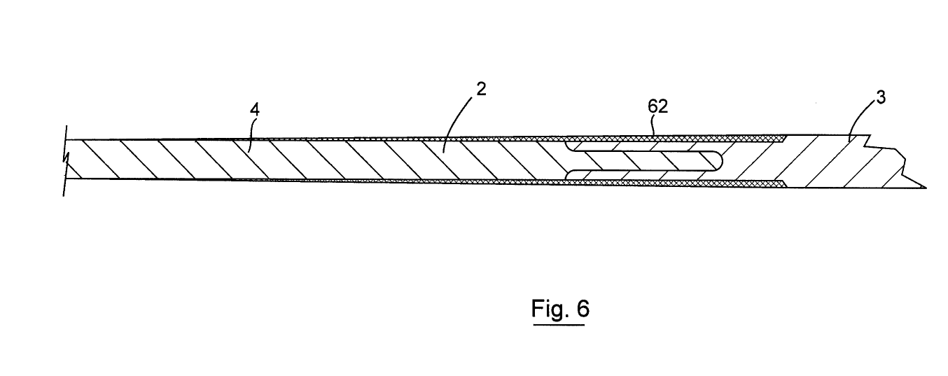

FIG. 6 illustrates a distally tapering sheath 62 which smoothens the transitions. The NiTi distal core 4 fits into a laser cut slot in the proximal SS core 3, or vice versa.

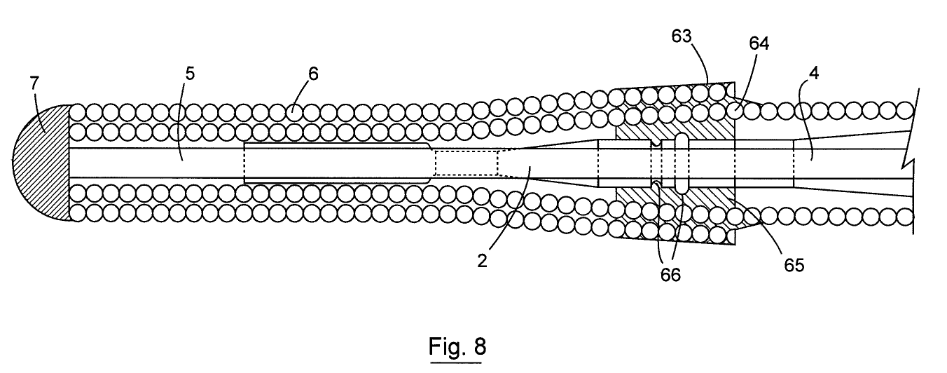

FIG. 8 illustrates recesses and/or ridges 66 which increase the step strength for example for retrieving or loading an embolic protection filter.

FIG. 9 illustrates the distally tapering NiTi core 4 which is a flattened section to offer gradual transition between the NiTi core 4 and the SS ribbon 5.

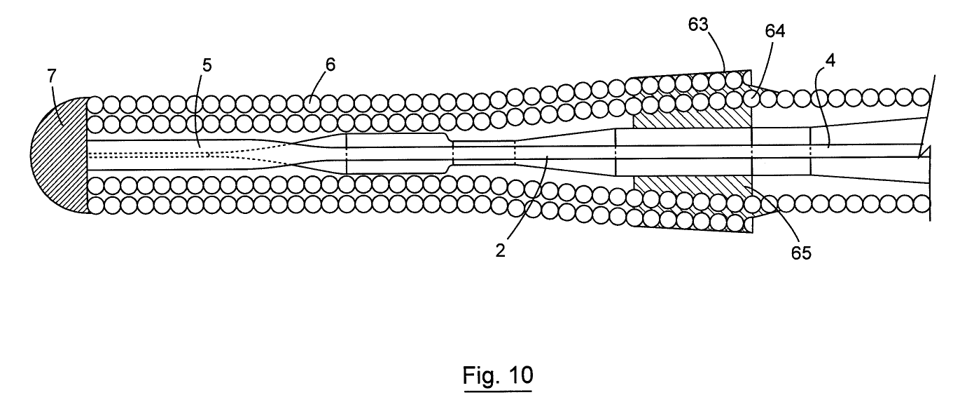

FIG. 10 illustrates opposite taper crossover to provide seamless transition.

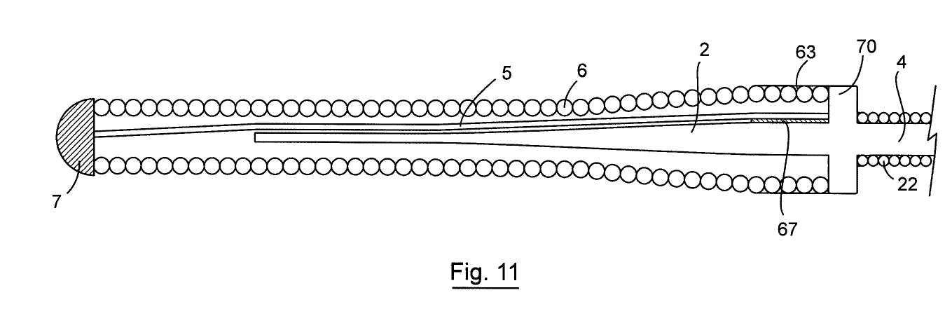

FIG. 11 illustrates an adhesive bond 67 to join the SS ribbon 5 to the NiTi core 4.

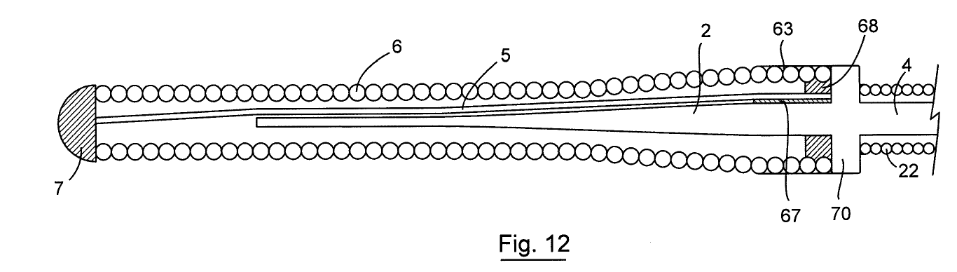

FIG. 12 illustrates an adhesive bond 68 to join the Pt coil 6 to the SS ribbon 5 to the NiTi core 4.

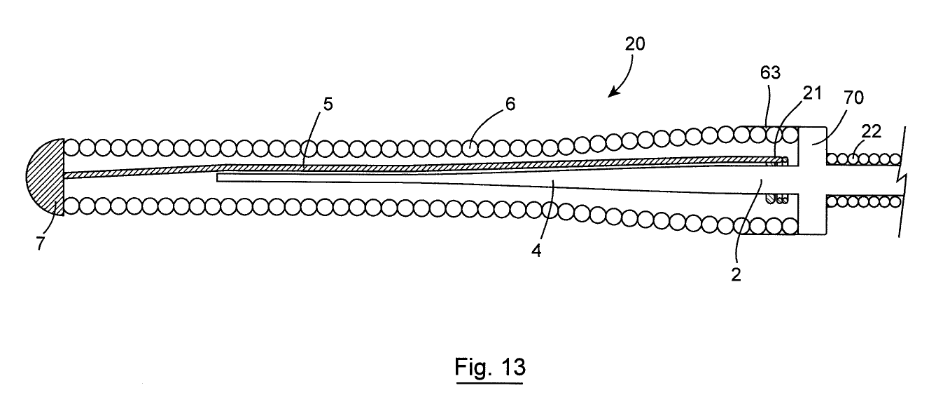

FIG. 13 illustrates another medical guidewire 20 according to the invention, which is similar to the medical guidewire 1 of FIGS. 1, 2 and 7, and similar elements in FIG. 13 are assigned the same reference numerals.

In this case the distal portion 5 extends around the circumference of the intermediate portion 4 in a spiral 21 to couple the distal portion 5 to the intermediate portion 4. The guidewire 20 comprises a second coil 22 located radially outwardly of the core element 2. The second coil 22 extends along part of the intermediate portion 4.

FIG. 13 illustrates the SS ribbon 5 coiled around the core 4 and fixed in place through an adhesive bond or a laser weld or a ridge ground to the NiTi core 4.

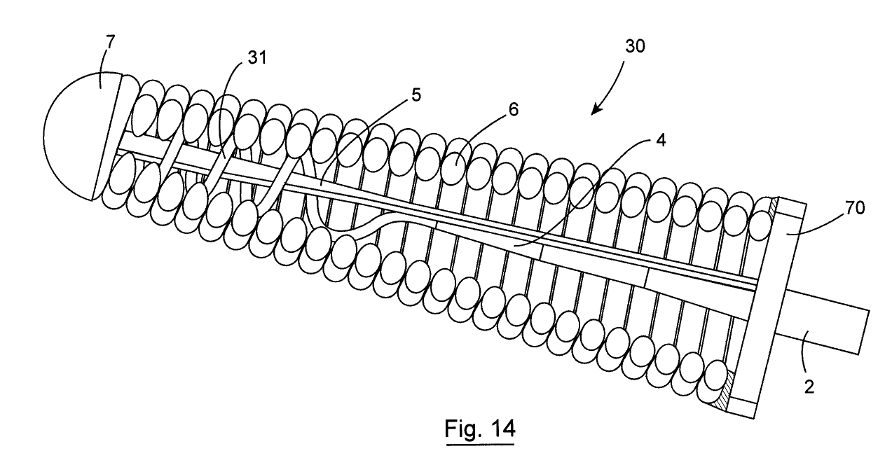

Referring to FIG. 14 there is illustrated another medical guidewire 30 according to the invention, which is similar to the medical guidewire 1 of FIGS. 1, 2 and 7, and similar elements in FIG. 14 are assigned the same reference numerals.

In this case the intermediate portion 4 extends around the circumference of the distal portion 5 in a spiral 31.

FIG. 14 illustrates the distal end of the NiTi core 4 coiled to remove resistance to bending. The coil 31 also helps to centre the NiTi core 4 in bending. The SS ribbon 5 tapers down proximally, providing a smooth transition where the NiTi core 4 is coiled.

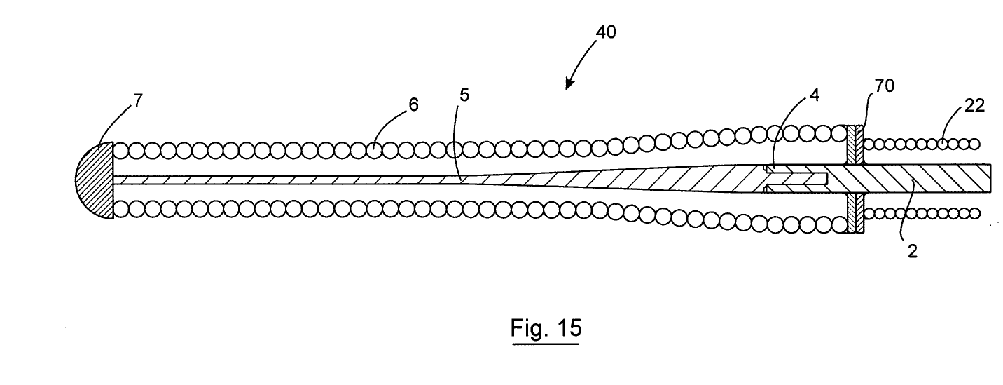

In FIG. 15 there is illustrated another medical guidewire 40 according to the invention, which is similar to the medical guidewire 20 of FIG. 13, and similar elements in FIG. 15 are assigned the same reference numerals.

In this case the distal portion 5 tapers distally radially inwardly.

FIG. 15 illustrates the proximal NiTi core 4 heated to expand over the distal SS core 5. When cooled, this arrangement provides attachment.

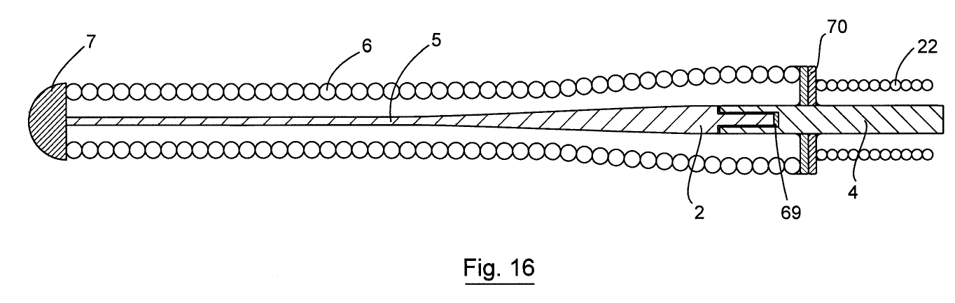

FIG. 16 illustrates an adhesive bond 69 to join the proximal NiTi core 4 to the distal SS core 5.

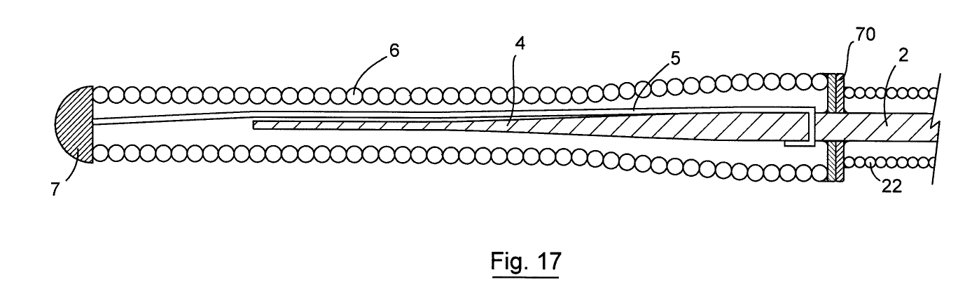

FIG. 17 illustrates the SS ribbon 5 slotted through the NiTi core 4.

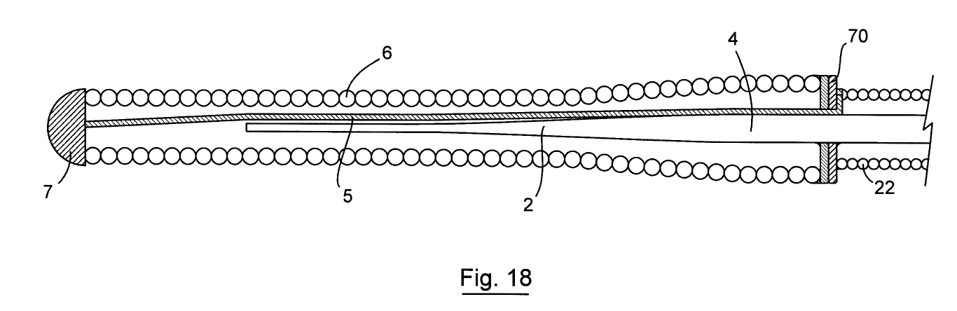

FIG. 18 illustrates an adjacent layer shim 70. The distal NiTi layer is laser welded to the NiTi core 4. The proximal SS layer is laser welded to the SS ribbon 5 and the SS coil 22.

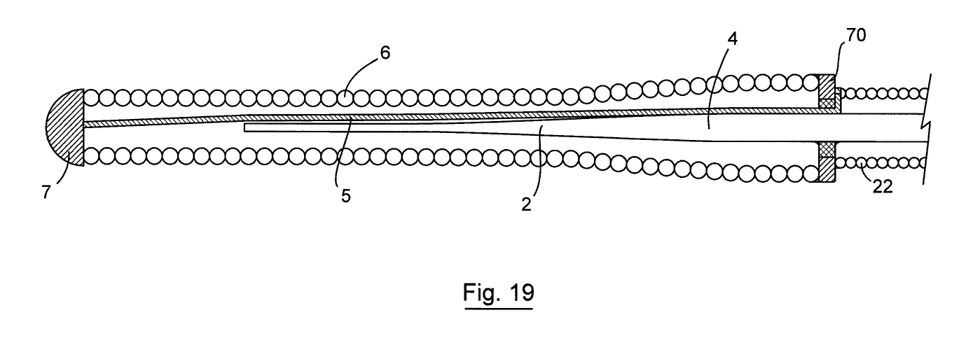

FIG. 19 illustrates the platinum coil 6 laser welded to the SS layer 70. The concave layer shim 70 has an inner NiTi layer laser welded to the NiTi core 4, and an outer SS layer laser welded to the SS ribbon 5 and the SS coil 22.

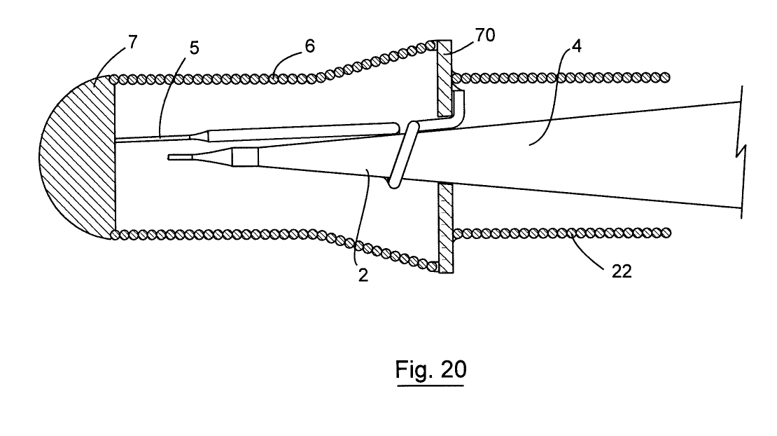

FIG. 20 illustrates the SS wire 5 coiled to provide a ridge, with a distal end flattened to aid steerability and shaping properties.

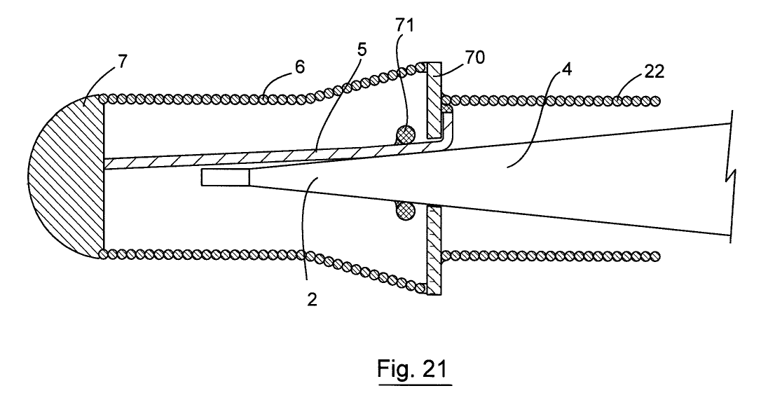

FIG. 21 illustrates a wire ridge 71 laser welded to the NiTi core 4. The SS ribbon 5 slots under the shim 70 and is attached thereto.

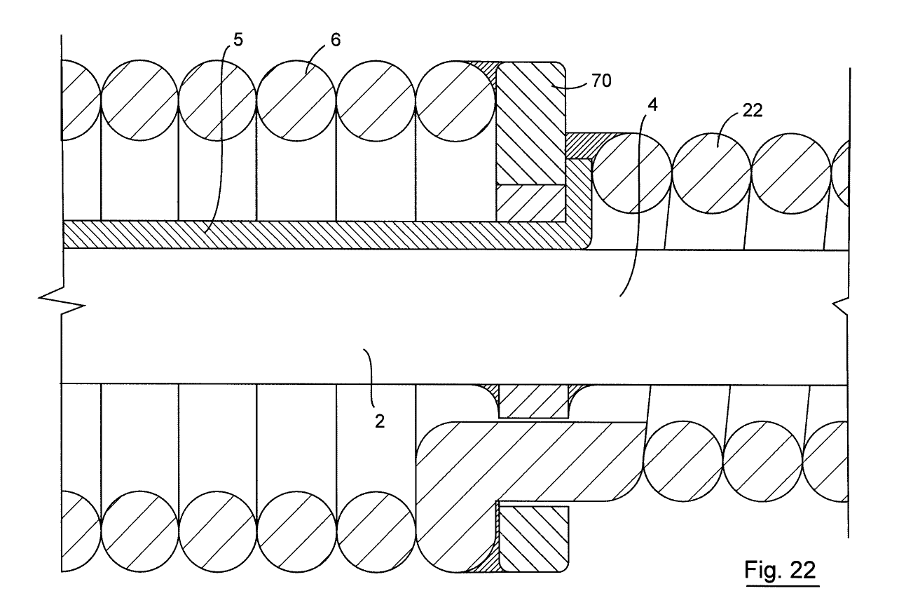

FIG. 22 illustrates the distal Pt coil 6 slotted through the shim 70 to provide a single proximal/distal coil.

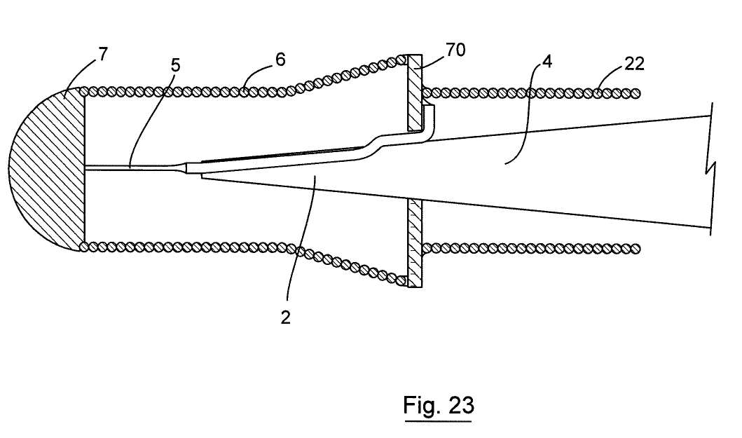

FIG. 23 illustrates the SS wire 5 slotted into a laser cut slot on the NiTi core 4. The distal end of the SS ribbon 5 is flattened to aid tip shaping and steerability properties.

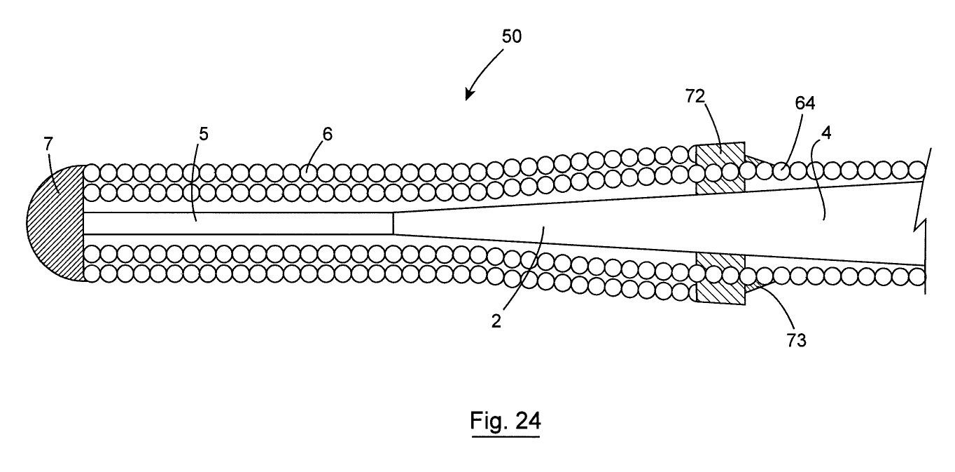

FIG. 24 illustrates another medical guidewire 50 according to the invention, which is similar to the medical guidewire 1 of FIGS. 1, 2 and 7, and similar elements in FIG. 24 are assigned the same reference numerals.

In this case the intermediate portion 4 is of stainless steel. The intermediate portion 4 is formed integrally with the distal portion 5, and is formed integrally with the proximal portion 3.

FIG. 24 illustrates the solder tip 7, the Pt outer coil 6, the SS inner coil 64, the SS core 2, the solder step 72, the solder fillet 73. The distal for example 1 cm of the core 5 may be flattened to improve tip shaping/steering properties.

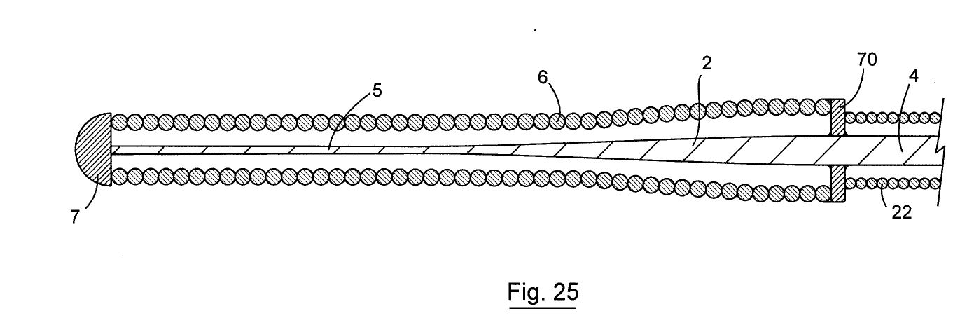

FIG. 25 illustrates the shim 70 (<1 mm length) laser welded to the SS core wire 4 to provide a step.

The distal end of the core 5 is flattened and either heat treated of chemically treated for example Nickel leach to remove super elastic properties. The step 70 may be provided by solder layers or a shim or a polymer mould.

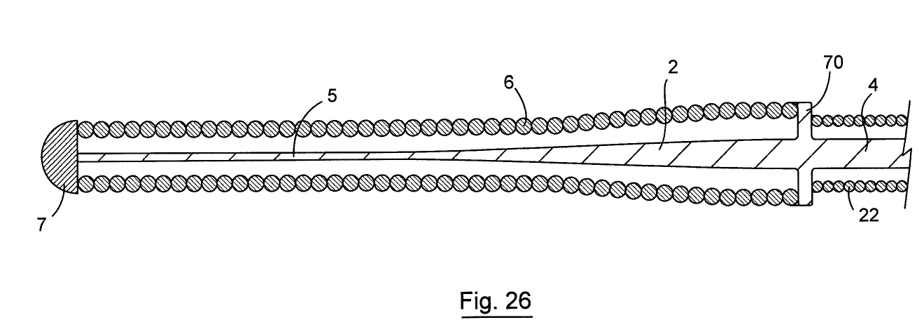

FIG. 26 illustrates the platinum coil 6, the step 70 ground from the SS core wire 4, the stainless steel coil 22, the coils 6, 22 laser welded or soldered to the step 70.

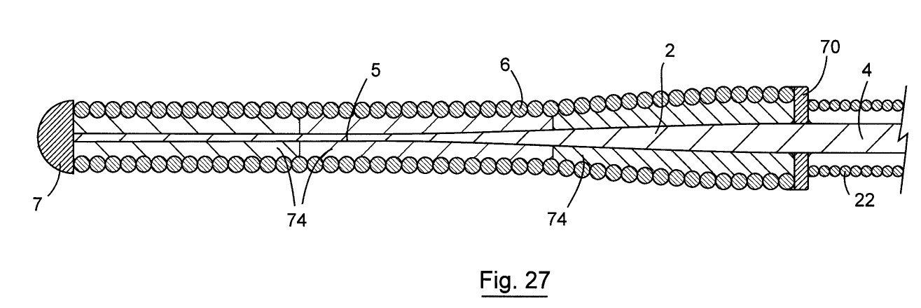

FIG. 27 illustrates multiple layers 74 of variable softness grade polymers which aid in defining distal tip flexibility. They may also be imparted with a radiopaque filler. This would allow the platinum outer coil 6 to be replaced with a SS coil or low friction sheath.

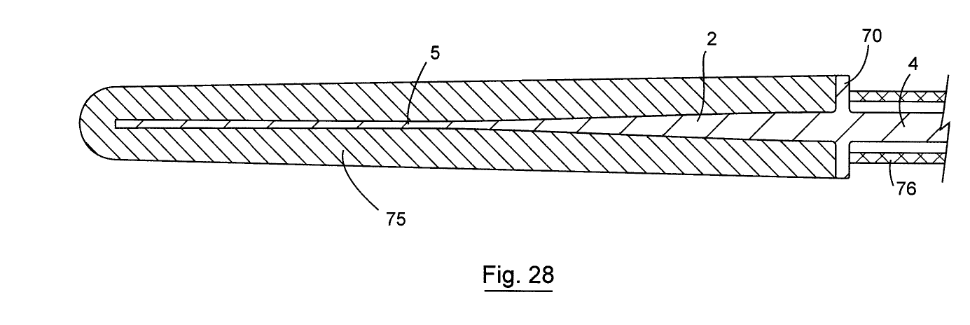

FIG. 28 illustrates a polymer 75 with a radiopaque filler, and a low friction polymer sheath 76. The polymer 75 may have low friction properties or a low friction coating may be applied.

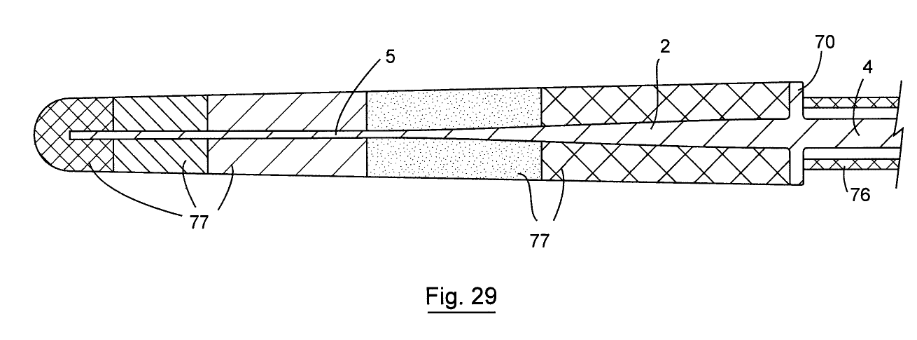

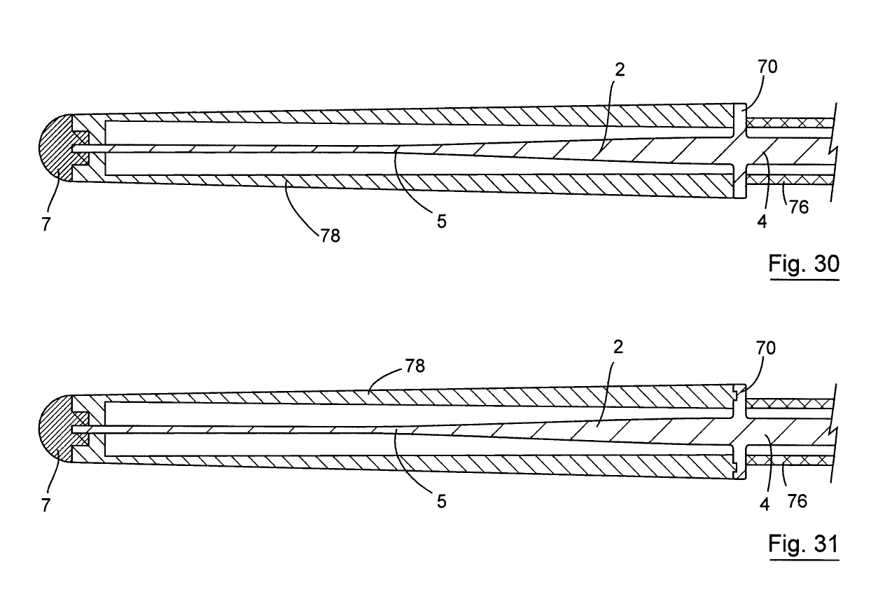

FIG. 29 illustrates layers 77 of variable stiffness polymers with a radiopaque filler. The polymer layers 77 may have low friction properties or a low friction coating may be applied.

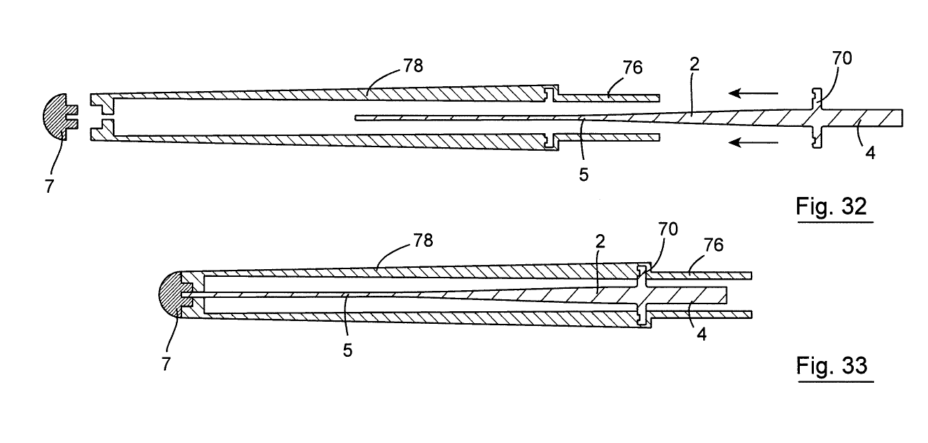

FIG. 32 illustrates a polymer sheath 78 imparted with a radiopaque filler. The sheath 78 has variable wall thickness to vary flexibility. The separate nose 7 allows for a denser application of radiopaque filler to maximise radiopacity. The sheath 78 can expand over the step 70 to aid manufacturability and anchor the sheath 78 to the guidewire 2. The sheath 78 and nose 7 are ultrasonically or heat welded to the core wire 2 and the step 70.

The invention is not limited to the embodiments hereinbefore described, with reference to the accompanying drawings, which may be varied in construction and detail.

1. A medical guidewire comprising:—
a core element extending along at least part of the length of the guidewire;
at least part of the core element being plastically deformable for steering of the guidewire.

2. A guidewire as claimed in claim 1 wherein the core element comprises a distal portion.

3. A guidewire as claimed in claim 2 wherein the distal portion is plastically deformable.

4. A guidewire as claimed in claim 3 wherein the distal portion is of stainless steel.

5. A guidewire as claimed in claim 2 wherein the distal portion tapers proximally radially inwardly.

6. A guidewire as claimed in claim 2 wherein the distal portion tapers distally radially inwardly.

7. A guidewire as claimed in claim 2 wherein the core element comprises an intermediate portion extending proximally of the distal portion.

8. A guidewire as claimed in claim 7 wherein the intermediate portion is coupled to the distal portion.

9. A guidewire as claimed in claim 7 wherein the intermediate portion overlaps the distal portion.

10. A guidewire as claimed in claim 9 wherein the distal end of the intermediate portion is located distally of the proximal end of the distal portion.

11. A guidewire as claimed in claim 7 wherein the intermediate portion tapers distally radially inwardly.

12. A guidewire as claimed in claim 7 wherein the intermediate portion extends around at least part of the circumference of the distal portion.

13. A guidewire as claimed in claim 12 wherein the intermediate portion extends in a spiral.

14. A guidewire as claimed in claim 7 wherein the distal portion extends around at least part of the circumference of the intermediate portion.

15. A guidewire as claimed in claim 14 wherein the distal portion extends in a spiral.

16. A guidewire as claimed in claim 7 wherein the intermediate portion is formed integrally with the distal portion.

17. A guidewire as claimed in claim 7 wherein the intermediate portion is formed separately from the distal portion.

18. A guidewire as claimed in claim 7 wherein the intermediate portion is of the same material as the distal portion.

19. A guidewire as claimed in claim 7 wherein the intermediate portion is of a different material to the distal portion.

20. A guidewire as claimed in claim 7 wherein the intermediate portion is of a shape-memory material.

21. A guidewire as claimed in claim 20 wherein the intermediate portion is of Nitinol.

22. A guidewire as claimed in claim 7 wherein the intermediate portion is of stainless steel.

23. A guidewire as claimed in claim 7 wherein the core element comprises a proximal portion extending proximally of the intermediate portion.

24. A guidewire as claimed in claim 23 wherein the proximal portion is coupled to the intermediate portion.

25. A guidewire as claimed in claim 23 wherein the proximal portion overlaps the intermediate portion.

26. A guidewire as claimed in claim 25 wherein the distal end of the proximal portion is located distally of the proximal end of the intermediate portion.

27. A guidewire as claimed in claim 23 wherein the intermediate portion tapers proximally radially inwardly.

28. A guidewire as claimed in claim 23 wherein the proximal portion is formed integrally with the intermediate portion.

29. A guidewire as claimed in claim 23 wherein the proximal portion is formed separately from the intermediate portion.

30. A guidewire as claimed in claim 23 wherein the proximal portion is of the same material as the intermediate portion.

31. A guidewire as claimed in claim 23 wherein the proximal portion is of a different material to the intermediate portion.

32. A guidewire as claimed in claim 23 wherein the proximal portion is of stainless steel.
